(19) United States
(12) Patent Application Publication
(10) Pub. No.: US 2026/0020831 A1
(43) Pub. Date: Jan. 22, 2026

(54) SUSPENSION SYSTEM AND IMAGING SYSTEM

(71) Applicant: GE Precision Healthcare LLC, Waukesha, WI (US)

(72) Inventors: Jianqiang Yang, Beijing (CN); Chunyu Wang, Beijing (CN); Shaobo Gu, Beijing (CN)

(21) Appl. No.: 19/266,563

(22) Filed: Jul. 11, 2025

(30) Foreign Application Priority Data
Jul. 19, 2024 (CN) 202410976855.1

(51) Int. Cl. A61B 6/00; A61B 6/10

(52) U.S. Cl. CPC A61B 6/4464; A61B 6/105; A61B 6/107; A61B 6/4476; A61B 6/547

(57) ABSTRACT

The present invention relates to a suspension system and an imaging system. The suspension system includes a rotating mechanism, a suspension column, and a carrying apparatus. The rotating mechanism includes a rotating member and a motor unit, and the motor unit is connected to the rotating member and drives the rotating member to rotate. The suspension column is provided with a first accommodating cavity, and the first accommodating cavity is used to accommodate at least a portion of the rotating mechanism. A first end of the carrying apparatus is connected to a bulb tube apparatus of an imaging device, and a second end thereof is connected to the rotating member.

### CROSS-REFERENCE TO RELATED APPLICATIONS

This application is based on and claims priority to Chinese Application No. 202410976855.1, filed on Jul. 19, 2024, the entire contents of which is herein incorporated by reference.

### TECHNICAL FIELD

The present invention relates to medical imaging technologies, and more specifically, to a suspension system for a medical imaging device and a medical imaging system.

### BACKGROUND

An X-ray medical imaging system relates to an imaging technology for obtaining internal structural information of a human body by using penetrative capability of X-rays, and is widely used in clinical diagnosis, disease detection, and treatment evaluation. The X-ray medical imaging system may include an X-ray tube (also referred to as a bulb tube apparatus), for generating X-rays and projecting the X-rays outward to pass through a body site of a patient. Due to varying absorption capabilities of different tissues of a human body for X-rays, the X-rays may experience different levels of attenuation when passing through the human body. These attenuated X-rays are then captured by an imaging detector for forming an image.

To better meet clinical needs and improve imaging quality and efficiency, a suspended X-ray imaging system is proposed. The suspended X-ray imaging system typically includes a robotic arm, which is capable of supporting and positioning an X-ray tube. The robotic arm is movable and/or rotatable, for the X-ray tube to project X-rays to a patient from different angles and positions, so as to provide a plurality of imaging angles of view.

At present, rotation of the robotic arm in the suspended X-ray imaging system can be implemented by means of manual operations to further drive the X-ray tube to rotate. This requires an operator to directly control the robotic arm to rotate by using a handle, joystick, or another mechanical apparatus. However, such a manual rotation manner may impose a large physical burden on the operator, and rotation accuracy and safety thereof cannot be guaranteed. In addition, the prior art also proposes to implement the rotation of the robotic arm in the suspended X-ray imaging system by means of electric power or a combination of electric power and manual power. However, the suspended X-ray imaging system that uses a motor to implement the rotation of the robotic arm currently on the market has a large size and is quite bulky, which limits the installation and use of the suspended X-ray imaging system in some space-limited application scenarios.

Therefore, it is desirable to improve the existing suspended X-ray imaging system, so as to overcome at least one of the disadvantages described above.

### SUMMARY OF THE INVENTION

The technical solutions proposed by the present invention are intended to solve one or more of the above-described problems of the existing suspended X-ray imaging system.

A first aspect of the present invention provides a suspension system, comprising: a rotating mechanism, comprising: a rotating member; and a motor unit, connected to the rotating member and driving the rotating member to rotate; a suspension column, provided with a first accommodating cavity, the first accommodating cavity being used to accommodate at least a portion of the rotating mechanism; and a carrying apparatus, a first end of which is connected to a bulb tube apparatus of an imaging device, and a second end of which is connected to the rotating member.

In at least one embodiment of the first aspect of the present invention, the suspension column comprises a side cover, which is detachably mounted to a side surface of the suspension column, so as to cover the first accommodating cavity.

In at least one embodiment of the first aspect of the present invention, the suspension column further comprises one or more fasteners, used to mount the side cover to the side surface of the suspension column.

In at least one embodiment of the first aspect of the present invention, the suspension system further comprises a position monitoring apparatus, used to monitor an angular position of the bulb tube apparatus relative to the suspension column.

In at least one embodiment of the first aspect of the present invention, the suspension system further comprises a braking apparatus, used to limit rotation of the bulb tube apparatus relative to the suspension column.

In at least one embodiment of the first aspect of the present invention, the suspension system further comprises a control unit, wherein the control unit is configured to be communicatively connected to the position monitoring apparatus and the braking apparatus and is used to: on the basis of the angular position of the bulb tube apparatus relative to the suspension column detected by the position monitoring apparatus, control the braking apparatus to limit the rotation of the bulb tube apparatus relative to the suspension column.

In at least one embodiment of the first aspect of the present invention, the braking apparatus comprises: a gear, fixedly connected to the suspension column; and a movable member, comprising: a body, mounted to the carrying apparatus; and a telescopic rod, located on one side of the body and capable of protruding outward or retracting inward relative to the body.

In at least one embodiment of the first aspect of the present invention, the control unit is further configured to: in response to determining that the angular position detected by the position monitoring apparatus is a target angular position, control the telescopic rod to protrude outward relative to the body, so as to enable the telescopic rod to be inserted between two adjacent teeth of the gear, thereby limiting the rotation of the bulb tube apparatus connected to the carrying apparatus relative to the suspension column; and in response to receiving a rotation instruction, control the telescopic rod to retract inward relative to the body, so as to allow the bulb tube apparatus connected to the carrying apparatus to freely rotate relative to the suspension column.

In at least one embodiment of the first aspect of the present invention, the telescopic rod is an electromagnetic telescopic rod.

In at least one embodiment of the first aspect of the present invention, the position monitoring apparatus comprises an encoder, comprising: a first encoding disk, fixedly connected to the suspension column; and a second encoding disk, mounted to the carrying apparatus.

In at least one embodiment of the first aspect of the present invention, the at least a portion accommodated within the first accommodating cavity comprises the motor unit, the second end of the carrying apparatus is provided with an accommodating portion, the accommodating portion comprises a second accommodating cavity, and the second accommodating cavity is used to accommodate at least a portion of the rotating member, the position monitoring apparatus, and the braking apparatus.

In at least one embodiment of the first aspect of the present invention, the carrying apparatus comprises a connecting member, located within the second accommodating cavity and used to connect the rotating member to the carrying apparatus.

In at least one embodiment of the first aspect of the present invention, the suspension system further comprises a bearing, located within the second accommodating cavity, an inner ring of the bearing is mounted to the suspension column, and an outer ring of the bearing is mounted to the carrying apparatus.

In at least one embodiment of the first aspect of the present invention, the carrying apparatus comprises a bottom cover, which is detachably mounted to a bottom surface of the carrying apparatus, so as to cover the second accommodating cavity.

In at least one embodiment of the first aspect of the present invention, the carrying apparatus further comprises an intermediate portion, located between the first end and the second end of the carrying apparatus, the intermediate portion is provided with a third accommodating cavity, and the third accommodating cavity is used to accommodate a circuit board.

In at least one embodiment of the first aspect of the present invention, the carrying apparatus further comprises a shielding cover, and the shielding cover is detachably mounted to a side surface of the intermediate portion of the carrying apparatus, so as to cover the third accommodating cavity.

In at least one embodiment of the first aspect of the present invention, the shielding cover is provided with a cable access port.

In at least one embodiment of the first aspect of the present invention, an upper end of the suspension column is connected to a ceiling, a lower end of the suspension column is provided with the first accommodating cavity, the suspension column further comprises a plurality of sleeves, and the plurality of sleeves are sequentially nested from bottom to top, and used to implement telescopic function of the suspension column.

A second aspect of the present invention provides an imaging system, comprising: the suspension system according to any one of the above-described paragraphs; and an imaging device, comprising a bulb tube apparatus.

In at least one embodiment of the second aspect of the present invention, the imaging device is an X-ray imaging device.

### BRIEF DESCRIPTION OF THE DRAWINGS

In order to further describe the previous and other advantages and features in the embodiments of the present invention, more detailed descriptions of the embodiments of the present invention will be presented with reference to the accompanying drawings. It should be understood that these accompanying drawings delineate only typical embodiments of the present invention, and thus will not be considered as a limitation on the scope of protection claimed by the present invention.

FIG. 1 shows an example application scenario of an imaging system according to an exemplary embodiment of the present disclosure.

FIG. 2 shows a schematic partial structural diagram of the imaging system according to an exemplary embodiment of the present disclosure.

FIG. 3 shows a schematic structural diagram of a suspension column of a suspension system in a fully extended state according to an exemplary embodiment of the present disclosure.

FIG. 4 shows a schematic partial structural diagram of the suspension system according to an exemplary embodiment of the present disclosure.

FIG. 5 shows a schematic partial structural exploded view of the suspension system according to an exemplary embodiment of the present disclosure.

FIG. 6 shows a schematic partial structural diagram of the suspension system in an assembled state according to an exemplary embodiment of the present disclosure.

FIG. 7 shows a schematic structural diagram of a braking apparatus according to an exemplary embodiment of the present disclosure.

FIG. 8 shows a schematic partial cross-sectional view of the suspension system in the assembled state according to an exemplary embodiment of the present disclosure.

FIG. 9 shows a schematic partial structural diagram of the suspension system viewed from the bottom according to an exemplary embodiment of the present disclosure.

FIG. 10 shows a schematic control logic block diagram of the suspension system according to an exemplary embodiment of the present disclosure.

### DETAILED DESCRIPTION

The present invention will be further described below with reference to specific embodiments and the accompanying drawings. More details are set forth in the following description in order to facilitate thorough understanding of the present invention, but it will be clear that the present invention can be implemented in many other forms other than those described herein, and those skilled in the art can, without departing from the essence of the present invention, make similar alterations and modifications according to practical applications. Therefore, the scope of protection of the present invention should not be limited by the content of the specific embodiments.

Specific terms have been used in the present application to describe the embodiments of the present application. For example, “an embodiment”, “another embodiment”, and/or “some embodiments” refer/refers to a certain feature, structure, or characteristic related to at least one embodiment of the present application. Therefore, it should be emphasized and noted that two or more references to “an embodiment”, “another embodiment”, or “some embodiments” in various places in this specification are not necessarily referring to the same embodiment. In addition, certain features, structures, or characteristics of one or more embodiments of the present application may be properly combined.

It should be noted that in the description of the embodiments of the present application, various features are sometimes incorporated into one embodiment, accompanying drawing, or description thereof in the present disclosure for the purpose of streamlining the descriptions disclosed in the present application and aiding in the understanding of one or more embodiments. However, this disclosure method does not mean that the present application object needs more features than the features mentioned in the claims.

In the descriptions of the present disclosure, it should be noted that directions or position relationships indicated by the terms “center”, “upper”, “lower”, “left”, “right”, “vertical”, “horizontal”, and the like described herein are based on the directions or position relationships shown by the accompanying drawings, which are used only for describing the present disclosure and for brevity of description, but do not indicate or imply that an indicated device or component must have a specific direction or must be constructed and operated in a specific direction. Therefore, this cannot be understood as a limitation on the present disclosure. In addition, the terms “first” and “second” are used only for descriptive purposes and cannot be construed as indicating or implying relative importance. In the descriptions of the present disclosure, it should be noted that, unless otherwise expressly specified and defined, the terms “installation”, “connect”, “connection”, and “coupling” should be understood in a broad sense, which, for example, may be a fixed connection or a detachable connection; may be a mechanical connection or an electrical connection; may be a direct connection or an indirect connection by means of an intermediate medium; or may be internal communication between two elements. Those of ordinary skill in the art can understand the specific meanings of the above-described terms in the present disclosure according to the specific situation.

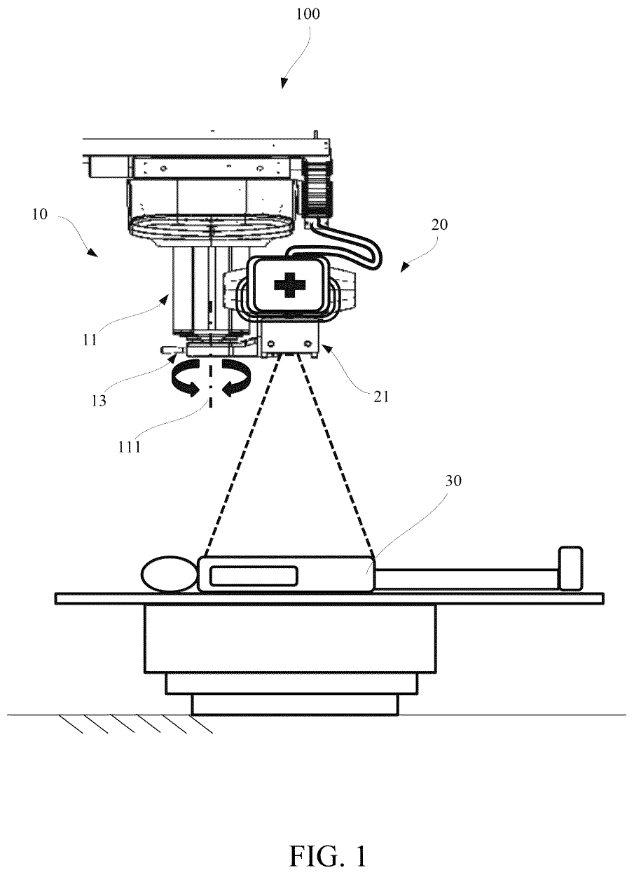

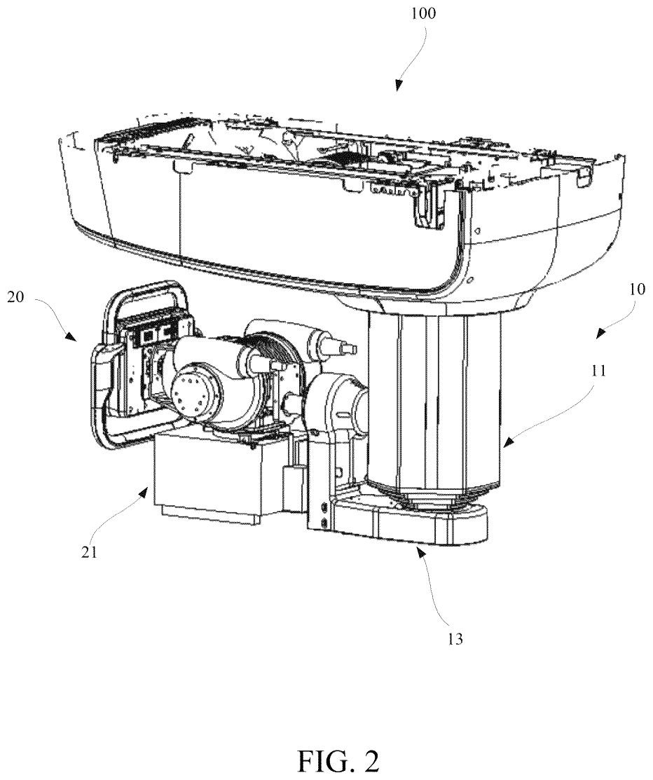

FIG. 1 shows an example application scenario of an imaging system 100 according to an exemplary embodiment of the present disclosure. FIG. 2 shows a schematic partial structural diagram of the imaging system 100 according to an exemplary embodiment of the present disclosure. As shown in FIG. 1 and FIG. 2, the imaging system 100 may include a suspension system 10 and an imaging device 20. The suspension system 10 may include a suspension column 11 and a carrying apparatus 13. One end (for example, the upper end) of the suspension column 11 may be connected directly or indirectly (for example, via a guide rail) to a ceiling or another fixed position, and the other end (for example, the lower end) may be connected to the carrying apparatus 13. A first end (the right end shown in FIG. 1, and the left end shown in FIG. 2) of the carrying apparatus 13 may be connected to a bulb tube apparatus 21 of the imaging device 20. When the carrying apparatus 13 is driven by a motor unit (not shown in FIG. 1 and FIG. 2) to rotate centering on a longitudinal axis 111 of the suspension column 11, the bulb tube apparatus 21 connected to the carrying apparatus 13 may also rotate centering on the longitudinal axis 111 of the suspension column 11, and rotate to a plurality of different positions relative to a patient 30. In this way, the imaging device 20 may provide a plurality of different imaging angles of view for imaging the patient 30. In some embodiments, the imaging device 20 may be an X-ray imaging device, or another imaging device including the bulb tube apparatus 21.

In some embodiments, the suspension column 11 may be configured such that the distance between two ends (for example, the upper end and the lower end shown in FIG. 1 and FIG. 2) of the suspension column 11 is adjustable, for example, the suspension column 11 may be configured to be telescopic. In this case, when one end (for example, the upper end) of the suspension column 11 is connected to the ceiling, the height of the lower end of the suspension column 11 from the ceiling is adjustable. In this way, the height of the bulb tube apparatus 21 connected to the suspension system 10 from the ceiling can be adjusted, and further the height of the bulb tube apparatus 21 from the patient 30 can be adjusted.

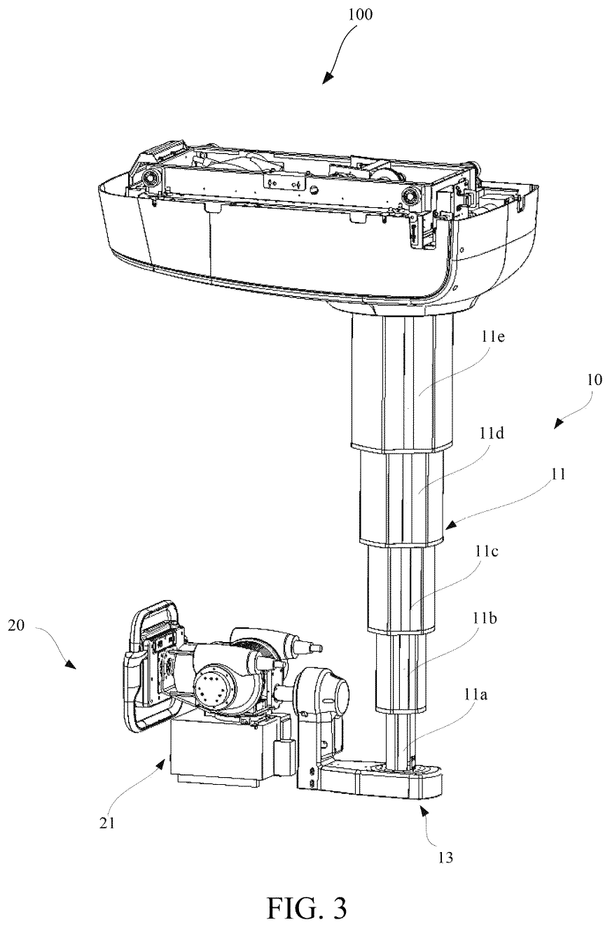

FIG. 3 shows a schematic structural diagram of a suspension column 11 of a suspension system 10 in a fully extended state according to an exemplary embodiment of the present disclosure. As shown in FIG. 3, the suspension column 11 may include a plurality of sleeves (for example, five sleeves are exemplarily shown in FIG. 3, respectively marked as 11a, 11b, 11c, 11d, and 11e). These sleeves may be sequentially nested from bottom to top. For example, the bottom sleeve 11a may be nested into the secondary bottom sleeve 11b, the secondary bottom sleeve 11b may be nested into the intermediate sleeve 11c, the intermediate sleeve 11c may be nested into the secondary top sleeve 11d, and the secondary top sleeve 11d may be nested into the top sleeve 11e. The carrying apparatus 13 may be connected to the lower end of the bottom sleeve 11a of the suspension column 11.

When the suspension column 11 is in an extended state (for example, a fully extended state, or a partially extended state), one or more of the nested sleeves may each extend from the sleeve above it. FIG. 3 shows a fully straightened state in which all of the sleeves (including the bottom sleeve 11a, the secondary bottom sleeve 11b, the intermediate sleeve 11c, and the second top sleeve 11d) that are nested each extends from the sleeve above it. When the suspension column 11 is in a retracted state, other sleeves except the top sleeve Ile can be nested or accommodated into the sleeve above it, as shown in FIG. 1 and FIG. 2. By means of enabling the suspension column 11 to be in different states (for example, a fully extended state, a partially extended state, or a retracted state), the carrying apparatus 13 connected to the bottom of the suspension column 11 (and the bulb tube apparatus 21 connected to the carrying apparatus 13) can be adjusted to different heights, so as to increase imaging flexibility of the imaging device 20.

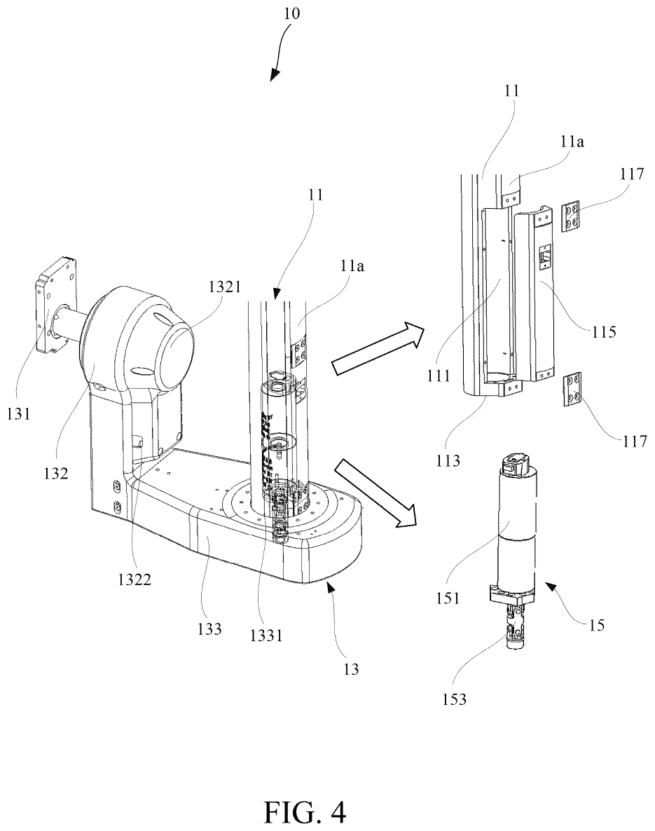

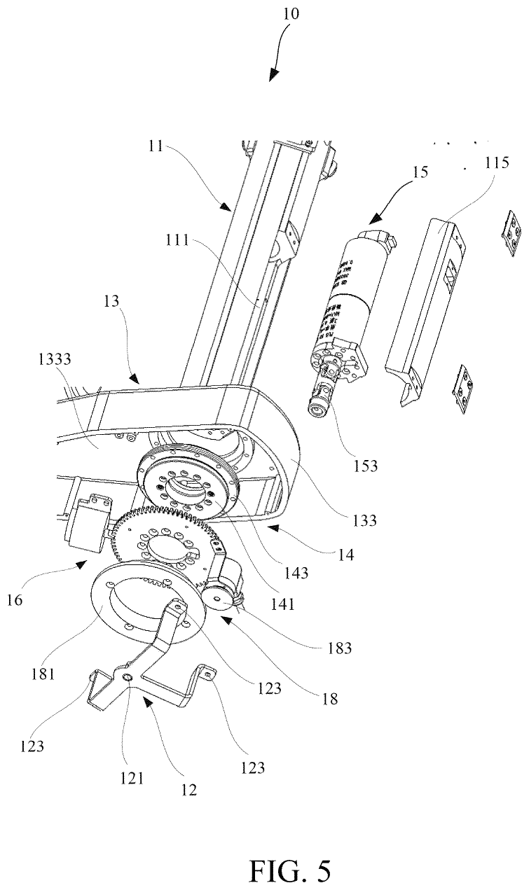

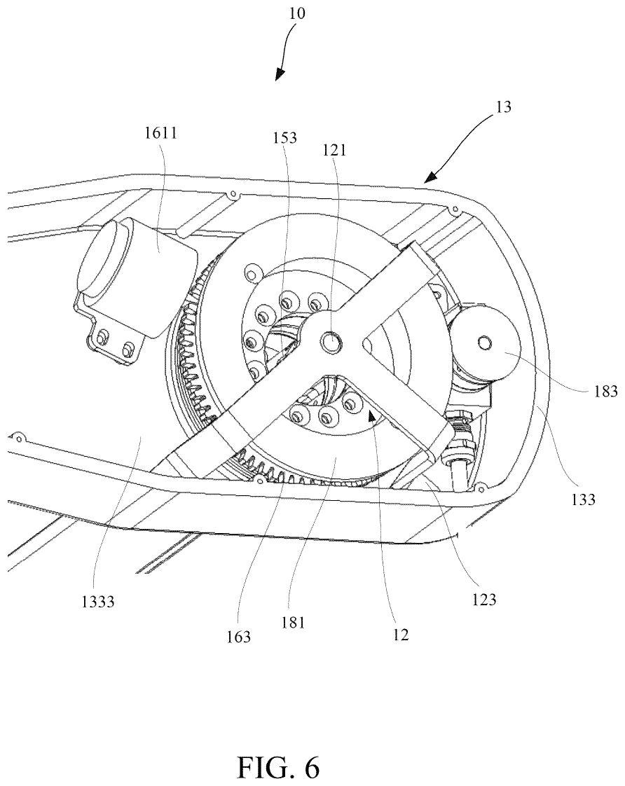

FIG. 4 shows a schematic partial structural diagram of the suspension system 10 according to an exemplary embodiment of the present disclosure. Referring to FIG. 4, the first end (the left end shown in FIG. 4) of the carrying apparatus 13 of the suspension system 10 may be provided with a connecting adapter 131. The connecting adapter 131 may be used to connect the carrying apparatus 13 to the bulb tube apparatus 21 of the imaging device 10. The second end (the right end shown in FIG. 4) of the carrying apparatus 13 may be provided with an accommodating portion 133. The accommodating portion 133 may be provided with an accommodating cavity 1333 (not shown in FIG. 4, referring to FIG. 5 and FIG. 6), and the accommodating cavity 1333 may be used to accommodate a connecting member 12, a bearing 14, a braking apparatus 16, and a position monitoring apparatus 18 (not shown in FIG. 4, referring to FIG. 5). Referring to the lower right of FIG. 4, the suspension system 10 may further include a rotating mechanism 15. The rotating mechanism 15 may include a motor unit 151 and a rotating member 153. The motor unit 151 may be connected to the rotating member 153 and drive the rotating member 153 to rotate. At least a portion of the rotating member 153 may be located within the accommodating cavity 1333 of the accommodating portion 133 of the carrying apparatus 13, and the rotating member 153 may be fixedly connected to the accommodating portion 133. Under the driving of the motor unit 151, the rotating member 153 can rotate and drive the carrying apparatus 13 fixedly connected to the rotating member 153 (and the bulb tube apparatus 21 fixedly connected to the carrying apparatus 13) to rotate.

Referring to the upper right of FIG. 4, the suspension column 11 may be provided with an accommodating cavity 111 at the bottom sleeve 11a of the suspension column 11, and the accommodating cavity 111 may be used to accommodate the motor unit 151, and optionally, accommodate a portion of the rotating member 153. The suspension column 11 may be provided with an opening 113 at the bottom of the suspension column 11, and the opening 113 may be in communication with the accommodating cavity 111. Referring to the left side of FIG. 4, a top portion of the accommodating portion 133 of the carrying apparatus 13 may be provided with an opening 1331, and the opening 1331 may be in communication with the accommodating cavity 1333 of the accommodating portion 133. When the suspension column 11, the carrying apparatus 13, and the rotating mechanism 15 are in an assembled state, as shown on the left side of FIG. 4, the motor unit 151 and optionally a portion of the rotating member 153 of the rotating mechanism 15 may be located within the accommodating cavity 111 of the suspension column 11, and at least a portion of the rotating member 153 may be located within the accommodating cavity 1333 of the accommodating portion 133 of the carrying apparatus 13 and fixedly connected to the accommodating portion 133. That is, when the suspension column 11, the carrying apparatus 13, and the rotating mechanism 15 are in an assembled state, the suspension column 11 and the carrying apparatus 13 may be connected to each other via the rotating mechanism 15 at the opening 113 of the suspension column 11 and the opening 1331 of the carrying apparatus 13.

In the suspension system 10 of the present invention, the accommodating cavity 111 is provided in the suspension column 11, and at least a portion of the rotating mechanism 15 (for example, the motor unit 151) is disposed or mounted within the accommodating cavity 111. Compared to mounting a motor outside the suspension column, the present invention can improve structural compactness of the suspension system 10 and reduce the size of the suspension system 10, so that structural compactness of the imaging system 100 can be improved and the size of the imaging system 100 can be reduced. In addition, the present invention further proposes that the accommodating cavity 111 is provided at the bottom of the suspension column 11, and at least a portion of the rotating mechanism 15 (for example, the motor unit 151) is mounted within the accommodating cavity 111, which in turn can allow the rotating mechanism 15 to directly drive the carrying apparatus 13 (and the bulb tube apparatus 21 fixedly connected to the carrying apparatus 13) to rotate. Compared to the solution in which the motor is mounted above the suspension column (such as between the suspension column and the guide rail above the suspension column), the present invention can eliminate the needs for a bearing and another transmission apparatus (for example, a synchronous belt or gear) having a large size and weight in the solution in which the motor is mounted above the suspension column, so that the overall weight and cost of the suspension system 10 can be greatly reduced, and further, safety of the suspension system 10 can be improved due to reduction of the overall weight.

In addition, as shown in FIG. 2 to FIG. 4, a connection direction of the carrying apparatus 13 and the suspension column 11 is a vertical direction (that is, the carrying apparatus 13 and the suspension column 11 are connected to each other in the vertical direction). In addition, as shown in FIG. 2 and FIG. 3, a connection direction of the carrying apparatus 13 and the bulb tube apparatus 21 is a horizontal direction (that is, the carrying apparatus 13 and the bulb tube apparatus 211 are connected to each other in the horizontal direction). The carrying apparatus 13 connects the suspension column 11 and the bulb tube apparatus 21 in two connection directions substantially perpendicular to each other, which can further improve the structural compactness of the imaging system 100 and reduce the size of the imaging system 100. It should be understood that the connection direction of the carrying apparatus 13 and the suspension column 11 and the connection direction of the carrying apparatus 13 and the bulb tube apparatus 21 shown in the figures are only exemplary, and are not intended to limit the scope of the present application. In other embodiments, the connection direction of the carrying apparatus 13 and the suspension column 11 may also be a horizontal direction. Correspondingly, the connection direction of the carrying apparatus 13 and the bulb tube apparatus 21 may be a vertical direction. In some other embodiments, the carrying apparatus 13 may connect the suspension column 11 and the bulb tube apparatus 21 in any other two connection directions (not limited to the two connection directions substantially perpendicular to each other).

Referring to the upper right of FIG. 4, in some embodiments, the suspension column 11 may include a side cover 115, and the side cover 115 may be detachably mounted to a side surface of the suspension column 11 to cover the accommodating cavity 111 of the suspension column 11. The suspension column 11 may further include one or more fasteners 117 (exemplarily shown as two fasteners 117 in FIG. 4), and the fasteners 117 may be used to mount the side cover 115 to the side surface of the suspension column 11. In an embodiment, as shown in FIG. 4, the fastener 117 may include a plate-shaped member. The plate-shaped member may be provided with a plurality of threaded holes (four threaded holes are exemplarily illustrated in FIG. 4). At least one threaded hole (two threaded holes are exemplarily illustrated in FIG. 4) may correspond to a corresponding threaded hole on the suspension column 11. At least another threaded hole (two threaded holes are exemplarily illustrated in FIG. 4) may correspond to a corresponding threaded hole on the side cover 115. In this embodiment, the fastener 117 may further include a plurality of bolts, and each bolt may cooperate with a corresponding threaded hole on the plate-shaped member and a corresponding threaded hole on the suspension column 11 or the side cover 115 to mount the side cover 115 to the side surface of the suspension column 11. In other embodiments, the side cover 115 may be directly mounted to the side surface of the suspension column 11 by means of a snap-fit mounting manner, for example, the side cover 115 may have a snap-fit mounting member, the side surface of the suspension column 11 may have a snap-fit mounting adapter, and the side cover 115 may be mounted to the side surface of the suspension column 11 by means of a snap-fit mounting between the snap-fit mounting member and the snap-fit mounting adapter. By means of providing a detachably mounted side cover 115 on the side surface of the suspension column 11, on the one hand, an operator can be allowed to conveniently mount the rotating mechanism 15 to the accommodating cavity 111 of the suspension column 11 from the side surface of the suspension column 11, so that mounting difficulty of the suspension system 10 can be reduced and mounting cost can be reduced. On the other hand, the operator can subsequently operate and/or maintain the rotating mechanism 15 within the accommodating cavity 11, so that operation cost and/or maintenance cost of the suspension system 10 is reduced.

In some other embodiments, the suspension column 11 may not include the side cover 115, and the rotating mechanism 15 may be mounted to the accommodating cavity 111 from the opening 113 at the bottom of the suspension column 11.

Referring to FIG. 4, the carrying apparatus 13 may further include an intermediate portion 132, and the intermediate portion 132 may be located between the connecting adapter 131 and the accommodating portion 133. The intermediate portion 132 may be provided with an accommodating cavity (not shown in FIG. 4), and the accommodating cavity may be used to accommodate a circuit board. The accommodating cavity of the intermediate portion 132 may be in communication with the accommodating cavity 1333 of the accommodating portion 133. Control cables for various components of the suspension system 10 (for example, the rotating mechanism 15, the braking apparatus 16, and/or the position monitoring apparatus 18, not shown in FIG. 4, referring to FIG. 5) may extend from the accommodating cavity 1333 of the accommodating portion 133 to the accommodating cavity of the intermediate portion 132. The carrying apparatus 13 may further include a shielding cover 1321, and the shielding cover 1321 may be detachably mounted to a side surface of the intermediate portion 132, so as to cover the accommodating cavity of the intermediate portion 132 from the side surface of the intermediate portion 132. On one hand, the shielding cover 1321 can conceal the circuit board within the accommodating cavity of the intermediate portion 132, and on the other hand, the shielding cover 1321 can also have a function of shielding and protecting the circuit board. The shielding cover 1321 may be provided with a cable access port 1322, for the control cables to pass through.

FIG. 5 shows a schematic partial structural exploded view of the suspension system 10 according to an exemplary embodiment of the present disclosure. FIG. 6 shows a schematic partial structural diagram of the suspension system 10 in an assembled state according to an exemplary embodiment of the present disclosure. Referring to FIG. 5 and as described above, the accommodating portion 133 of the carrying apparatus 13 may be provided with an accommodating cavity 1333. The accommodating cavity 1333 may accommodate at least a portion of the rotating member 153 of the rotating mechanism 15. The suspension system 10 may include a connecting member 12, and the connecting member 12 may be placed within the accommodating cavity 1333 of the carrying apparatus 13 and used to connect the rotating member 153 to the carrying apparatus 13. In some embodiments, as shown in FIG. 5, the connecting member 12 may be configured in a claw shape, and the claw-shaped connecting member 12 may be provided with an intermediate connecting portion 121 and a plurality of end connecting portions 123 (three end connecting portions 123 are exemplarily illustrated in FIG. 5). Referring to FIG. 6, the intermediate connecting portion 121 may be connected to the bottom of the rotating member 153, and the end connecting portions 123 may be connected to a cavity wall of the accommodating portion 133 of the carrying apparatus 13. When the suspension system 10 is in an assembled state, the intermediate connecting portion 121 of the connecting member 12 may be connected and fitted with the rotating member 153 (for example, by means of bolts), and the end connecting portions 123 of the connecting member 12 may be connected and fitted with the cavity wall of the accommodating portion 133 of the carrying apparatus 13 (for example, by means of bolts). In this way, the rotating member 153 can be connected to the carrying apparatus 13, so that the carrying apparatus 13 (and the bulb tube apparatus 21 connected to the carrying apparatus 13) can be driven to rotate by the motor unit 151. In the present application, the connecting member 12 may be designed to have a claw structure, and the claw structure allows the connecting member 12 to be provided with a plurality of end connecting portions 123 in the circumferential direction to be connected and fitted with the carrying apparatus 13, so that on one hand, the connection strength between the connecting member 12 and the carrying apparatus 13 can be enhanced, and further the connection strength between the rotating member 153 and the carrying apparatus 13 can be enhanced; and on the other hand, the plurality of end connecting portions 123 arranged in the circumferential direction can effectively transfer rotational kinetic energy of the rotating member 153 to the carrying apparatus 13, so that power performance of the motor unit 151 of the rotating mechanism 15 can be improved.

Referring to FIG. 5, the suspension system 10 may further include a bearing 14, which may be provided with an inner ring 141 and an outer ring 143. When the suspension system 10 is in an assembled state, the inner ring 141 may be mounted (for example, by means of bolts) to the bottom of the suspension column 11, and the outer ring 143 may be mounted (for example, by means of bolts) to the cavity wall of the accommodating portion 13 of the carrying apparatus 13. In this way, under the driving of the rotating member 153, the carrying apparatus 13 (and the bulb tube apparatus 21 connected to the carrying apparatus 13) may rotate around the suspension column 11. A center portion of the bearing 14 may be provided with a through hole, which may allow the bottom of the rotating member 153 to pass therethrough. In some embodiments, the bearing 14 may be a flange cross roller bearing. Compared to other bearings, the use of a flange cross roller bearing can reduce the number of parts required for the assembly of the bearing, simplify the assembly process, improve quality consistency, reduce the space occupied by the bearing 14 within the accommodating cavity 1333, and reduce the total cost.

Referring to FIG. 5, the suspension system 10 may further include a braking apparatus 16, and the braking apparatus 16 may be used to limit rotation of the carrying apparatus 13 (and the bulb tube apparatus 21 connected to the carrying apparatus 13) relative to the suspension column 11.

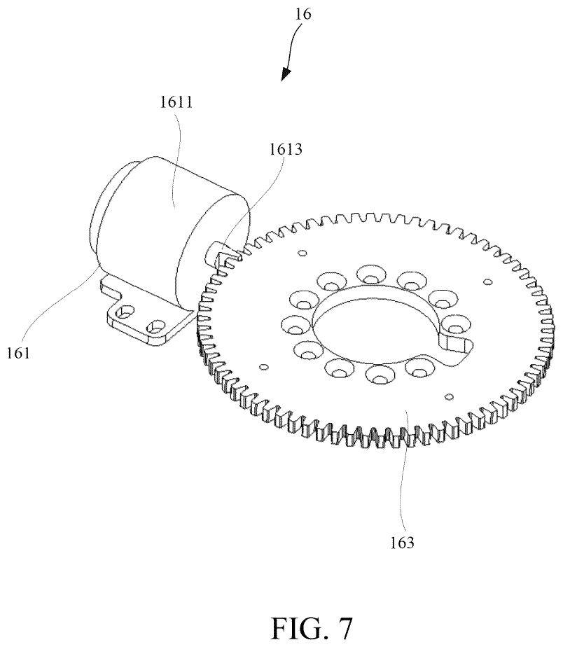

FIG. 7 shows a schematic structural diagram of a braking apparatus 16 according to an exemplary embodiment of the present disclosure. In some embodiments, the braking apparatus 16 may include a movable member 161 and a gear 163. The movable member 161 may include a body 1611 and a telescopic rod 1613. The body 1611 may be mounted to the carrying apparatus 13, for example, mounted to the cavity wall of the accommodating portion 13 of the carrying apparatus 13 by means of bolts (as shown in FIG. 6). The telescopic rod 1613 can be located on one side of the body 1611 and can protrude outward or retract inward relative to the body 1611. In FIG. 7, a state in which the telescopic rod 1613 protrudes outward with respect to the body 1611 is illustrated. In some embodiments, the telescopic rod 1613 may be an electromagnetic telescopic rod, and the electromagnetic telescopic rod can implement telescopic movement of the electromagnetic telescopic rod relative to the body 1611 by using the principle of electromagnetic induction. A center portion of the gear 163 may be provided with a through hole, which may allow the bottom of the rotating member 153 to pass therethrough. The gear 163 may be provided with a plurality of teeth at the circumference, and the distance between every two adjacent teeth among the teeth may be designed to allow the telescopic rod 1613 to be inserted between the two adjacent teeth. The gear 163 may be fixedly connected relative to the suspension column 11. For example, the gear 163 may be mounted to the bottom of the suspension column 11 by a means of bolts. During the rotation of the carrying apparatus 13 (and the bulb tube apparatus 21 connected to the carrying apparatus 13) around the suspension column 11, the movable member 161 can rotate with the rotation of the carrying apparatus 13, and the gear 163 can be fixed. When the telescopic rod 1613 of the movable member 161 protrudes outward and is inserted between two adjacent teeth of the gear 163, the movable member 161 may stop rotating under the action of the fixed gear 163, and further the carrying apparatus 13 (and the bulb tube apparatus 21 connected to the carrying apparatus 13) may also stop rotating accordingly. In this way, the braking apparatus 16 can limit the rotation of the bulb tube apparatus 21 relative to the suspension column 11.

Referring to FIG. 5, the suspension system 10 may further include a position monitoring apparatus 18, and the position monitoring apparatus 18 may be used to monitor an angular position of the carrying apparatus 13 (and the bulb tube apparatus 21 connected to the carrying apparatus 13) relative to the suspension column 11. In some embodiments, the position monitoring apparatus 18 may be an encoder, which may include a first encoding disk 181 and a second encoding disk 183, as shown in FIG. 5. The first encoding disk 181 may be fixedly connected relative to the suspension column 11. For example, as shown in FIG. 6, the first encoding disk 181 may be mounted to a gear 163 fixedly connected to the bottom of the suspension column 11 by means of bolts, so that the first encoding disk 181 is fixedly connected relative to the suspension column 11. The second encoding disk 183 may be mounted to the carrying apparatus 13. For example, as shown in FIG. 6, the second encoding disk 183 may be mounted to the cavity wall of the accommodating portion 13 of the carrying apparatus 13 by means of bolts. During the rotation of the carrying apparatus 13 (and the bulb tube apparatus 21 connected to the carrying apparatus 13) around the suspension column 11, the second encoding disk 183 can rotate with the rotation of the carrying apparatus 13, and the first encoding disk 181 can be fixed. It should be understood that, based on the position detection principle of the encoder (due to the fact that the position detection technology is well known to a person skilled in the art, details are not described herein again), the first encoding disk 181 and the second encoding disk 183 provided above may be used to implement monitoring of the angular position of the carrying apparatus 13 (and the bulb tube apparatus 21 connected to the carrying apparatus 13) relative to the suspension column 11. When the space within the accommodating cavity 1333 allows, the transmission ratio between the first encoding disk 181 and the second encoding disk 183 can be designed to be as large as possible, so that position detection accuracy of the position monitoring apparatus 18 is increased and position detection error is reduced. As shown in FIG. 5, a center portion of the first encoding disk 181 may be provided with a through hole, which may allow the bottom of the rotating member 153 to pass therethrough.

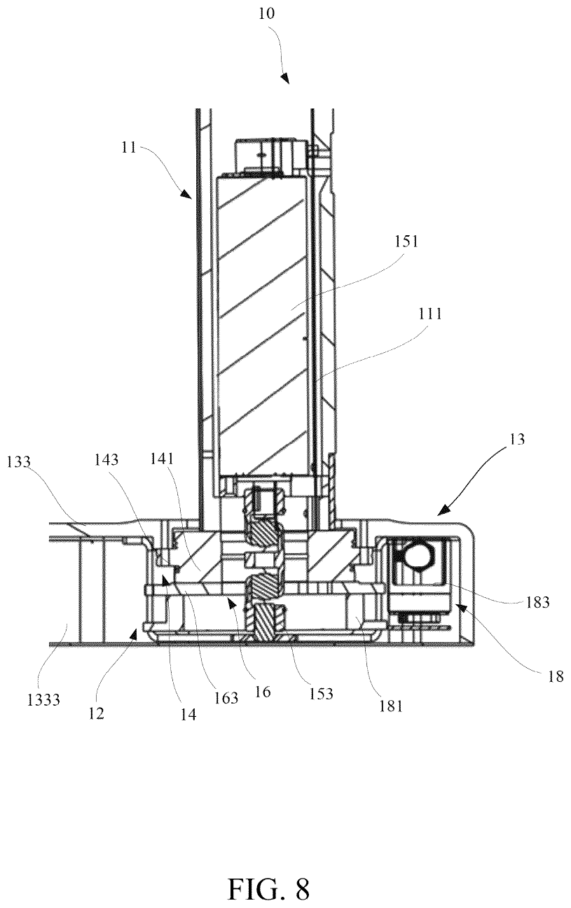

FIG. 8 shows a schematic partial cross-sectional view of the suspension system 10 in the assembled state according to an exemplary embodiment of the present disclosure. As shown in FIG. 8, when the suspension system 10 is in an assembled state, the motor unit 151 in the rotating mechanism 15 may be located within the accommodating cavity 111 of the suspension column 11, and the rotating member 153 located below the motor unit 151 may sequentially pass through the through hole in the center portion of the bearing 14, the through hole in the center portion of the gear 163 of the braking apparatus 16, and the through hole in the center portion of the first encoding disk 181 of the position monitoring apparatus 18, and is fixedly connected to the connecting member 12. As shown in FIG. 8, the connecting member 12, the bearing 14, the braking apparatus 16, and the position monitoring apparatus 18 may be located within the accommodating cavity 1333 of the carrying apparatus 13. The inner ring 141 of the bearing 14, the gear 163 in the braking apparatus 16, and the first encoding disk 181 in the position monitoring apparatus 18 may be sequentially mounted from top to bottom and fixedly connected relative to the suspension column 11. However, it should be understood that the upper and lower positional relationship among the inner ring 141, the gear 163, and the first encoding disk 181 may be adjusted, for example, the inner ring 141 may be located below the gear 163 and/or the first encoding disk 181, or the first encoding disk 181 may be located above the inner ring 141 and/or the gear 163, as long as the inner ring 141, the gear 163, and the first encoding disk 181 are fixedly connected relative to the suspension column 11. Again referring to FIG. 8, the connecting member 12, the outer ring 143 of the bearing 14, the movable member 161 (not shown in FIG. 8) in the braking apparatus 16, and the second encoding disk 183 in the position monitoring apparatus 18 may be fixedly connected (for example, fixedly connected by means of bolts) to the cavity wall of the accommodating portion 133 of the carrying apparatus 13.

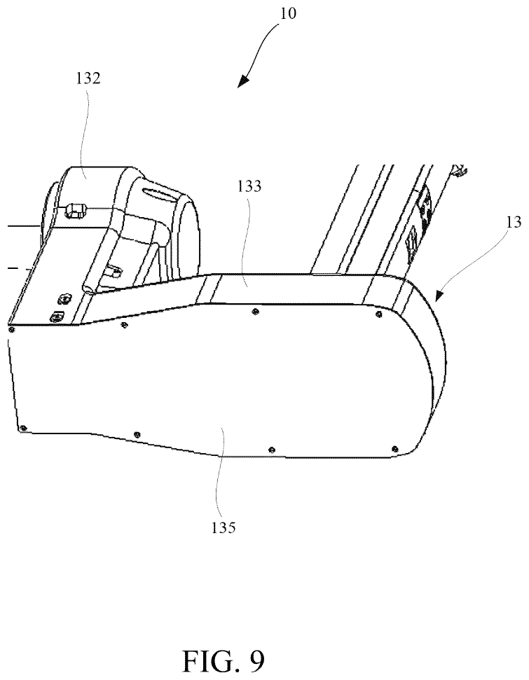

Referring to FIG. 9, FIG. 9 shows a schematic partial structural diagram of the suspension system 10 viewed from the bottom according to an exemplary embodiment of the present disclosure. As shown in FIG. 9, the carrying apparatus 13 may further include a bottom cover 135, which may be detachably mounted to the bottom surface of the carrying apparatus 13, so as to cover the accommodating cavity 1333 of the accommodating portion 133 and the accommodating cavity of the intermediate portion 132 of the carrying apparatus 13. By means of providing the detachably mounted bottom cover 135 on the bottom of the carrying apparatus 13, a plurality of benefits can be provided. For example, during the assembly of the suspension system 10, the detachably mounted bottom cover 135 may allow an operator to conveniently mount various components (for example, the connecting member 12, the bearing 14, the braking apparatus 16, the position monitoring apparatus 18) of the suspension system 10 within the accommodating cavity 1333 of the carrying apparatus 13 and perform wiring operations on the control cables for various components (for example, the rotating mechanism 15, the braking apparatus 16, and/or the position monitoring apparatus 18) of the suspension system 10, so that difficulty and cost of the assembly of the suspension system 10 can be reduced. In addition, during subsequent maintenance of the suspension system 10, the detachably mounted bottom cover 135 may also allow the operator to quickly and conveniently maintain various components in the accommodating cavity 1333, so that not only maintenance efficiency of the suspension system 10 can be improved, but also maintenance cost of the suspension system 10 can be reduced.

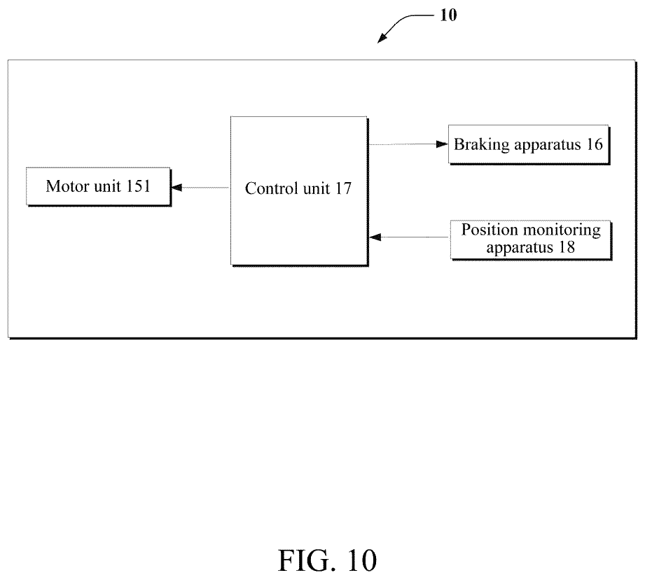

Referring to FIG. 10, FIG. 10 shows a schematic control logic block diagram of the suspension system 10 according to an exemplary embodiment of the present disclosure. The arrow direction in FIG. 10 may indicate a flow direction of information or a control signal.

As shown in FIG. 10, the suspension system 10 may further include a control unit 17. The control unit 17 may be used to be communicatively connected to the motor unit 151 and may be configured to: in response to receiving a rotation instruction in an electric mode, control the motor unit 151 to operate, so as to drive the rotating member 153 connected to the motor unit 151 to rotate, and further to drive the carrying apparatus 13 fixedly connected to the rotating member 153 (and the bulb tube apparatus 21 fixedly connected to the carrying apparatus 13) to rotate relative to the suspension column 11.

The control unit 17 may also be used to be communicatively connected to the position monitoring apparatus 18 and the braking apparatus 16, and may be configured to: on the basis of the angular position of the bulb tube apparatus 21 relative to the suspension column 11 detected by the position monitoring apparatus 18, control the braking apparatus 16 to limit the rotation of the bulb tube apparatus 21 relative to the suspension column 11. In some embodiments, the operation of the control unit 17 controlling the braking apparatus 16 to limit the rotation of the bulb tube apparatus 21 relative to the suspension column 11 may be performed in an electric mode (in the electric mode, the motor unit 151 may be used to drive the bulb tube apparatus 21 to rotate). When the motor unit 151 is used to drive the bulb tube apparatus 21 to rotate relative to the suspension column 11 and rotate to a target angular position in the electric mode, the control unit 17 may control the motor unit 151 to stop operating while controlling the braking apparatus 16 to limit the rotation of the bulb tube apparatus 21 relative to the suspension column 11 as described above. As described above, the braking apparatus 16 may include a movable member 161 and a gear 163, and the movable member 161 may include a body 1611 and a telescopic rod 1613. The control unit 17 may be further configured to: in response to determining that the angular position detected by the position monitoring apparatus 18 is a target angular position in the electric mode, control the telescopic rod 1613 of the movable member 161 to protrude outward relative to the body 1611, so as to enable the telescopic rod 1613 to be inserted between two adjacent teeth of the gear 163, and further to limit the rotation of the bulb tube apparatus 21 connected to the carrying apparatus 13 relative to the suspension column 11.

The control unit 17 may be further configured to: in response to receiving a rotation instruction, control the telescopic rod 1613 to retract inward relative to the body 1611, so as to allow the bulb tube apparatus 21 connected to the carrying apparatus 13 to freely rotate relative to the suspension column 11. In some embodiments, the rotation instruction may be received by the control unit 17 in the electric mode. When receiving the rotation instruction in the electric mode, the control unit 17 may control the motor unit 151 to operate while or after controlling the telescopic rod 1613 to retract inward relative to the body 1611 as described above, so as to drive the rotating member 153 connected to the motor unit 151 to rotate, and further to drive the carrying apparatus 13 fixedly connected to the rotating member 153 (and the bulb tube apparatus 21 fixedly connected to the carrying apparatus 13) to rotate relative to the suspension column 11. In other embodiments, the rotation instruction may be received by the control unit 17 in a manual mode (in the manual mode, the operator may manually rotate the bulb tube apparatus 21). When receiving the rotation instruction in the manual mode, the control unit 17 may control the telescopic rod 1613 to retract inward relative to the body 1611 as described above, so as to allow the operator to manually rotate the bulb tube apparatus 21.

For example, the control unit 17 may include: a circuit implemented by an application specific integrated circuit (ASIC), or a circuit implemented by a programmable logic device (PLD), for example, a field programmable gate array (FPGA), a microcontroller unit (MCU), or a digital signal processor (DSP), etc., Still for example, the control unit 17 may include a central processing unit (CPU).

Although the present invention has been described in accordance with preferred embodiments of the present disclosure, the present invention is not limited thereto but subject only to a limitation of the scope set forth in the appended claims. It should be understood by those skilled in the art that various modifications and changes may be made to the embodiments described herein without departing from the broader spirit and scope of the present invention as set forth in the appended claims.

1. A suspension system, characterized by comprising:
a rotating mechanism, comprising:
a rotating member; and
a motor unit, connected to the rotating member and driving the rotating member to rotate;
a suspension column, provided with a first accommodating cavity, the first accommodating cavity being used to accommodate at least a portion of the rotating mechanism; and
a carrying apparatus, a first end of which is connected to a bulb tube apparatus of an imaging device, and a second end of which is connected to the rotating member.

2. The suspension system according to claim 1, wherein the suspension column comprises a side cover, which is detachably mounted to a side surface of the suspension column, so as to cover the first accommodating cavity.

3. The suspension system according to claim 2, wherein the suspension column further comprises one or more fasteners, used to mount the side cover to the side surface of the suspension column.

4. The suspension system according to claim 1, further comprising a position monitoring apparatus, used to monitor an angular position of the bulb tube apparatus relative to the suspension column.

5. The suspension system according to claim 4, further comprising a braking apparatus, used to limit rotation of the bulb tube apparatus relative to the suspension column.

6. The suspension system according to claim 5, further comprising a control unit, wherein the control unit is configured to be communicatively connected to the position monitoring apparatus and the braking apparatus and is used to: on the basis of the angular position of the bulb tube apparatus relative to the suspension column detected by the position monitoring apparatus, control the braking apparatus to limit the rotation of the bulb tube apparatus relative to the suspension column.

7. The suspension system according to claim 6, wherein the braking apparatus comprises:
a gear, fixedly connected relative to the suspension column; and
a movable member, comprising:
a body, mounted to the carrying apparatus; and
a telescopic rod, located on one side of the body and capable of protruding outward or retracting inward relative to the body.

8. The suspension system according to claim 7, wherein the control unit is further configured to:
in response to determining that the angular position detected by the position monitoring apparatus is a target angular position, control the telescopic rod to protrude outward relative to the body, so as to enable the telescopic rod to be inserted between two adjacent teeth of the gear, thereby limiting the rotation of the bulb tube apparatus connected to the carrying apparatus relative to the suspension column; and
in response to receiving a rotation instruction, control the telescopic rod to retract inward relative to the body, so as to allow the bulb tube apparatus connected to the carrying apparatus to freely rotate relative to the suspension column.

9. The suspension system according to claim 7, wherein the telescopic rod is an electromagnetic telescopic rod.

10. The suspension system according to claim 7, wherein the position monitoring apparatus comprises an encoder, comprising:
a first encoding disk, fixedly connected relative to the suspension column; and
a second encoding disk, mounted to the carrying apparatus.

11. The suspension system according to claim 5, wherein the at least a portion accommodated within the first accommodating cavity comprises the motor unit,
the second end of the carrying apparatus is provided with an accommodating portion, the accommodating portion comprises a second accommodating cavity, and the second accommodating cavity is used to accommodate at least a portion of the rotating member, the position monitoring apparatus, and the braking apparatus.

12. The suspension system according to claim 11, wherein the carrying apparatus comprises a connecting member, located within the second accommodating cavity and used to connect the rotating member to the carrying apparatus.

13. The suspension system according to claim 11, further comprising a bearing, located within the second accommodating cavity,
an inner ring of the bearing being mounted to the suspension column, and an outer ring of the bearing being mounted to the carrying apparatus.

14. The suspension system according to claim 11, wherein the carrying apparatus comprises a bottom cover, which is detachably mounted to a bottom surface of the carrying apparatus, so as to cover the second accommodating cavity.

15. The suspension system according to claim 11, wherein the carrying apparatus further comprises an intermediate portion, located between the first end and the second end of the carrying apparatus, the intermediate portion is provided with a third accommodating cavity, and the third accommodating cavity is used to accommodate a circuit board.

16. The suspension system according to claim 15, wherein the carrying apparatus further comprises a shielding cover, and the shielding cover is detachably mounted to a side surface of the intermediate portion of the carrying apparatus, so as to cover the third accommodating cavity.

17. The suspension system according to claim 16, wherein the shielding cover is provided with a cable access port.

18. The suspension system according to claim 1, wherein an upper end of the suspension column is connected to a ceiling, and a lower end thereof is provided with the first accommodating cavity; and
the suspension column further comprises a plurality of sleeves, which are sequentially nested from bottom to top, and are used to implement telescopic function of the suspension column.
